(19) United States
(12) Patent Application Publication
(10) Pub. No.: US 2014/0123980 A1
(43) Pub. Date: May 8, 2014

(54) Automated Hypoxia Recovery System

(71) Applicants: Daniel Jean Rissacher, Potsdam, NY (US); James J. Carroll, Potsdam, NY (US)

(72) Inventors: Daniel Jean Rissacher, Potsdam, NY (US); James J. Carroll, Potsdam, NY (US)

(73) Assignee: CLARKSON UNIVERSITY, Potsdam, NY (US)

(21) Appl. No.: 14/073,202

(22) Filed: Nov. 6, 2013

Related U.S. Application Data
(60) Provisional application No. 61/723,033, filed on Nov. 6, 2012.

(51) Int. Cl. A61M 16/00; A61M 16/20; A61M 16/06; A61B 5/1455; A61B 5/00

(52) U.S. Cl. CPC A61M 16/0051; A61B 5/14552; A61M 16/0057; A61B 5/4836; A61M 16/0683; A61M 16/20; A61B 5/7405; A61B 5/747; A61B 5/7282
USPC 128/204.23

(57) ABSTRACT

Disclosed are methods and systems for detecting and remedying a potential hypoxic state. A wearable hypoxic state detector includes an SpO₂ sensor configured to measure a user's oxygen saturation, an oxygen reservoir, an oxygen conduit positioned to deliver oxygen from the oxygen storage reservoir to the user's inhalation flow path, and a controller. The controller is operably connected between the SpO₂ sensor and the oxygen delivery component, and is configured to automatically induce a flow of oxygen from the oxygen reservoir through the oxygen conduit when a predetermined oxygen saturation level is detected by the SpO₂ sensor.

### CROSS-REFERENCE TO RELATED APPLICATIONS

This application claims priority to U.S. Provisional Patent Application Ser. No. 61/723,033, filed on Nov. 6, 2012 and entitled “Automated Hypoxia Recovery System,” the entire disclosure of which is incorporated herein by reference.

### BACKGROUND

The present invention relates to systems and methods for life support, and, more specifically, to systems and methods for hypoxia recovery.

People are sometimes exposed to conditions where reduced availability of oxygen can cause hypoxia, where the body is deprived of sufficient oxygen resulting in decreased physical and mental capacity. Supplemental oxygen systems can prevent and/or correct hypoxia; however these systems can fail or may require human control of the system which can lead to problems due to human error. In addition, human control can become impossible after the onset of hypoxia either due to lack of recognition of the hypoxic state or the physical inability to control an oxygen system due to hypoxia itself.

The cardiopulmonary system's overall ability to deliver oxygen to the body can be monitored using a pulse oximeter which typically measures the absorption of red and infrared light through a patient's tissue to determine oxygen saturation (SpO₂) level. The pulse oximeter generally comprises at least one red light source and one infrared light source with a corresponding detector for each. The orientation of the source and detector can either be on opposing sides of the tissue (transmittance) or on the same surface (reflectance). De-oxyhemoglobin (RHb) absorbs more red light than oxyhemoglobin (HbO₂) and oxyhemoglobin absorbs more infrared light than de-oxyhemoglobin. Thus using this known relationship the oxygen saturation can be calculated. In addition, the absorption varies as blood vessels expand and contract allowing a pulse oximeter to also measure heart rate.

Life support systems are used in environments where reduced oxygen may be a concern. These systems may also include emergency backup systems or may be emergency systems themselves when oxygen is not being continuously applied. Typically these systems involve either human control or automatic control using some metric other than SpO₂ level. Thus, there is a continued need for systems and methods of identifying and remedying a hypoxic state and controlling supplemental oxygen using SpO₂ level monitoring.

### BRIEF SUMMARY

The present disclosure is directed to methods and apparatus for remedying a hypoxic state and automatically controlling supplemental oxygen using SpO₂ level monitoring. For example, a device that identifies a hypoxic state and regulates supplemental oxygen can include a reflectance or transmittance SpO₂ sensor, a controller, an oxygen reserve, and a method of delivering oxygen to the mouth or nose. This device could be used in civilian and military aviation as a primary or backup oxygen delivery system, or to treat medical conditions in a clinical setting, ambulance, military field trauma care, automated oxygen delivery first aid kit, or home use by someone requiring supplemental oxygen, among many other uses.

According to one embodiment is a wearable hypoxic state detection device. The device includes: (i) an SpO₂ sensor configured to measure a user's oxygen saturation; (ii) an oxygen reservoir; (iii) an oxygen conduit positioned to deliver oxygen from the oxygen storage reservoir to the user's inhalation flow path; and (iv) a controller operably connected between the SpO₂ sensor and the oxygen delivery component, wherein the controller is configured to automatically induce or modify a flow of oxygen from the oxygen reservoir through the oxygen conduit when a predetermined oxygen saturation level is detected by the SpO₂ sensor.

According to an aspect, the SpO₂ sensor is a reflectance or a transmittance sensor.

According to another aspect, the controller is configured to automatically stop the flow of oxygen from the oxygen reservoir through the oxygen conduit when a predetermined oxygen saturation level is detected by the SpO₂ sensor.

According to an aspect, the wearable hypoxic state detection device further includes a microphone.

According to an aspect, the wearable hypoxic state detection device further includes a speaker.

According to another aspect, the device is at least substantially worn on the user's head.

According to another aspect, the oxygen conduit is situated within a mask.

According to another aspect, the controller is operably connected to an oxygen flow valve.

According to an aspect, the wearable hypoxic state detection device further includes a communications module.

According to an aspect, the wearable hypoxic state detection device further includes an altimeter operably connected to the controller.

According to one embodiment is a hypoxic state detection system. The system comprises: (i) an oxygen reservoir; and (i) a wearable hypoxic state detection device comprising: (a) an SpO₂ sensor configured to measure a user's oxygen saturation; (b) an oxygen conduit positioned to deliver oxygen from the oxygen storage reservoir to the user's inhalation flow path; and (c) a controller operably connected between the SpO₂ sensor and the oxygen delivery component, wherein the controller is configured to automatically induce or modify a flow of oxygen from the oxygen reservoir through the oxygen conduit when a predetermined oxygen saturation level is detected by the SpO₂ sensor.

According to an aspect, the SpO₂ sensor is a reflectance or a transmittance sensor.

According to another aspect, the controller is configured to automatically stop the flow of oxygen from the oxygen reservoir through the oxygen conduit when a predetermined oxygen saturation level is detected by the SpO₂ sensor.

According to an aspect, the wearable hypoxic state detection device further includes a microphone.

According to an aspect, the wearable hypoxic state detection device further includes a speaker.

According to another aspect, the device is at least substantially worn on the user's head.

According to another aspect, the oxygen conduit is situated within a mask.

According to another aspect, the controller is operably connected to an oxygen flow valve.

According to an aspect, the wearable hypoxic state detection device further includes an altimeter operably connected to the controller.

According to one embodiment is a wearable hypoxic state detection device configured to be worn at least substantially on a user's head. The device includes: (i) an altimeter; (ii) an SpO₂ sensor configured to measure a user's oxygen saturation; (iii) an oxygen reservoir; (iv) an oxygen conduit positioned to deliver oxygen from the oxygen storage reservoir to the user's inhalation flow path; and (v) a controller in communication with the altimeter, and operably connected between the SpO₂ sensor and the oxygen delivery component, wherein the controller is configured to automatically induce or modify a flow of oxygen from the oxygen reservoir through the oxygen conduit when a predetermined oxygen saturation level or altitude is detected by the SpO₂ sensor, and wherein the controller is further configured to automatically stop the flow of oxygen from the oxygen reservoir through the oxygen conduit when a predetermined oxygen saturation level or altitude is detected by the SpO₂ sensor.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 illustrates a schematic of a wearable hypoxic detection and recovery device according to an embodiment;

FIG. 2 illustrates a schematic of a wearable hypoxic detection and recovery device according to an embodiment; and

FIG. 3 illustrates a flow chart of a method for detecting and remedying a hypoxic state using a wearable hypoxic detection and recovery device according to an embodiment.

### DETAILED DESCRIPTION

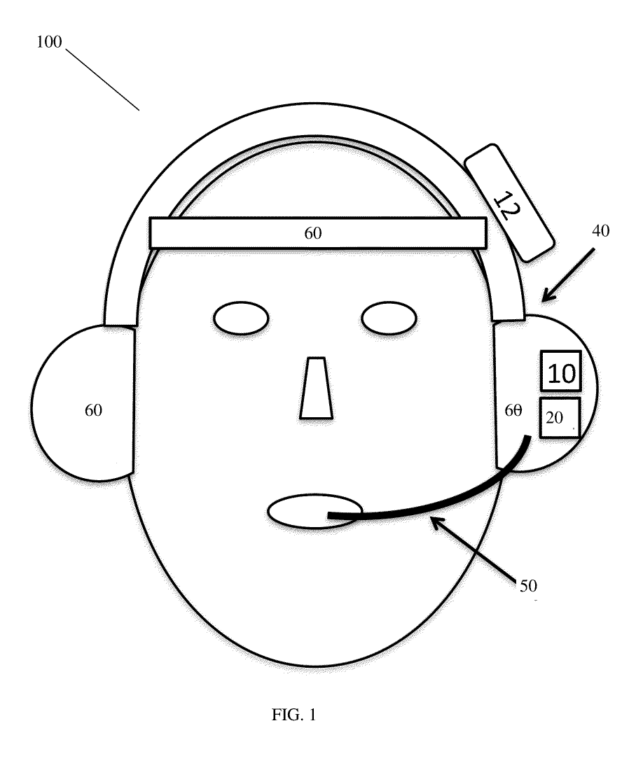

Referring now to the drawings, wherein like reference numerals refer to like parts throughout, there is seen in FIG. 1 an embodiment of an automated hypoxia recovery system 100. According to this embodiment, the wearable headset device 40 includes at least one reflectance or transmittance SpO₂ sensor 60. Sensor 60 could be located at one or more of several places. For example, sensor 60 could be located at or near the forehead for measuring SpO₂ levels using reflectance, or near one or both ears to measure SpO₂ levels by transmittance or reflectance. Alternatively, the sensor 60 can be remote from the wearable headset device 40 and transmits SpO₂ level measurements or data to the device via wired or wireless communication.

Wearable headset device 40 also includes a controller 20. The controller 20 is operably connected between the SpO₂ sensor 60 and the oxygen delivery components. The controller is programmed and/or configured to receive or request SpO₂ sensor data from SpO₂ sensor 60, modify or interpret that data, and either maintain the status quo or regulate oxygen delivery. For example, controller 20 can be programmed and/or configured to activate oxygen delivery only upon receipt of certain SpO₂ sensor data below a preprogrammed or predetermined threshold. As another example, controller 20 can be programmed and/or configured to deactivate oxygen delivery when a certain SpO₂ sensor data is then achieved, signaling the end of a need for supplemental oxygen. As another example, controller 20 can be programmed and/or configured to regulate the delivery of a specific oxygen flow rate which is dependent upon the specific SpO₂ level. For example, if the SpO₂ level is determined by SpO₂ sensor 60 to be at or below a certain predetermined threshold, then controller 20 can send a wired or wireless signal to the oxygen delivery components to deliver supplemental oxygen at or above a specific flow rate. Alternatively, controller 20 can send a wired or wireless signal to the oxygen delivery components to deliver air containing a certain percentage of oxygen.

The controller 20 can be programmed or configured with an adaptive algorithm according to an embodiment. This adaptive algorithm allows for many different users to utilize the wearable device 40. For example, the adaptive algorithm can include variables such as a baseline SpO₂ measurement, which can vary depending on the individual, temperature, time of day or year, location, and/or the altitude, etc. The variables can also include altitude of the individual. There are also many other possible variables. According to an embodiment, a baseline SpO₂ measurement is obtained prior to movement, takeoff, diving, etc., and can be triggered by, for example, powering on of the vehicle, device, etc., or by movement, or via a user interface. The device can also consider altitude, in which an altimeter or the altitude data is utilized to determine that high altitude conditions exist (or lack of cabin pressure in a pressurized aircraft) for both activation of supplemental oxygen and to provide a warning of loss of cabin pressure. The algorithm can factor the baseline SpO₂ measurement and/or altitude into the decision-making process, and/or into determining a minimum SpO₂ measurement for triggering a warning or for applying supplemental oxygen.

According to an embodiment, the wearable device 40 employs a multi-step process for remedying a hypoxic state. As an initial step, the device detects a possible or imminent hypoxic state (as indicated by low or decreasing SpO₂ levels), which triggers a warning to the user. The warning can be an audible, visual, and/or tactile warning. For example, the warning can be a light, a sound, an instrument reading, or a vibration, among other things. With the triggering of the warning, the device can set a certain amount of time in which the user can remedy the situation themselves, such as decreasing altitude, activating aircraft oxygen, etc. If that amount of time expires and the SpO₂ levels have not improved—or if the user bypasses the time period and requests immediate supplemental oxygen—the device can be triggered to induce or increase the supply of supplemental oxygen.

Wearable headset device 40 also includes oxygen delivery components configured and/or adapted to deliver supplemental oxygen to the wearer. According to an embodiment, the oxygen delivery components include an oxygen storage component 12 to store the supplemental oxygen. The oxygen may be, for example, compressed and stored in an oxygen storage component 12. According to the embodiment depicted in FIG. 1, the oxygen storage component 12 is affixed to the wearer. However, according to another embodiment, the oxygen bottle is remote from one or more of the other components of the system 100. For example, the oxygen storage component 12 may be stored in a container or storage device located within the transportation vehicle or gear worn by the user. As an example, the oxygen storage component 12 may be stored in a bottle located within a remote, and more secure, portion of an airplane with tubing that leads from the bottle to the vicinity of the user. The user can then simply connect the device 40 to the tubing, thereby allowing for the delivery of oxygen from the oxygen storage component 12 to the user when necessary.

As another embodiment, the user wears or carries the oxygen storage component 12. For example, the oxygen can be stored in a bottle or container that is directly incorporated into the wearable headset device 40. As another example, the oxygen can be stored in a bottle or container that is carried in a backpack by the user.

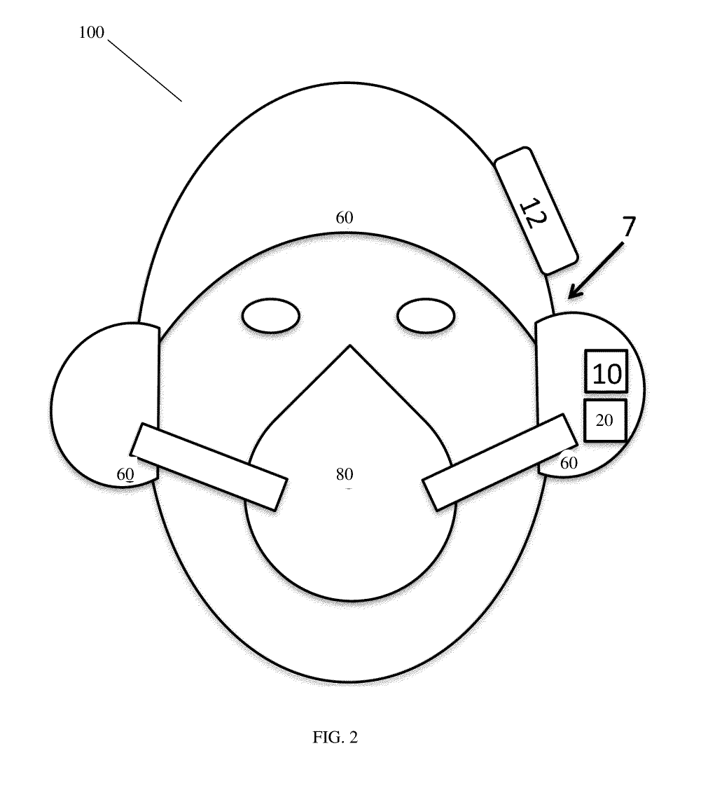

According to an embodiment, the oxygen delivery components include a device or system to deliver the oxygen from the oxygen storage component 12 to the user's nose and/or mouth. In FIG. 1, the oxygen delivery components include a boom 50 that has an oxygen delivery tube running from the oxygen storage component 12 to the user's mouth. The boom 50 may also include a microphone or other components. For example, in an aviation setting, the boom 50 can include a microphone for communication purposes. Similarly, headset 40 can include one or more speakers for communication purposes. According to another embodiment, shown in FIG. 2, the oxygen delivery components include a mask 80 that has an oxygen delivery tube running from the oxygen storage component 12 to the user's mouth.

According to an embodiment, wearable device 40 includes an electronically actuated valve or other mechanism to open, close, or regulate the flow of air from the oxygen storage component 12 to the user's mouth. The valve is operably connected to controller 20, which sends a wired or wireless signal to the value to open, close, or regulate the flow of air from the oxygen storage component 12 to the user's mouth.

According to an embodiment, wearable device 40 includes a communications module to communicate SpO₂ sensor data or levels, altitude, or other data from the device to a local receiver. The communications module can utilize any form of communications (including, for example, wireless, optical, or wired) and/or protocol (including, for example, WLAN, Wi-Fi, Internet-based communications, Bluetooth, and/or SMS, among others). Accordingly, wearable device 40 may interface or communicate via any connectivity or protocol (including, for example, wired, wireless, electrical and/or optical, as described above, as well as all forms of USB and/or removable memory).

According to an embodiment, the wearable headset device 40 also includes an altimeter 10 configured and/or adapted to monitor altitude of the device in aviation applications. For example, altimeter 10 is operably connected to the controller 20, which is programmed and/or configured to receive or request sensor data from altimeter 10, modify or interpret that data, and either maintain the status quo or regulate oxygen delivery. For example, controller 20 can be programmed and/or configured to activate oxygen delivery only upon receipt of certain altitude data above or below a predetermined threshold. As another example, controller 20 can be programmed and/or configured to deactivate oxygen delivery when a certain altitude is then achieved, signaling the end of a need for supplemental oxygen. As another example, controller 20 can be programmed and/or configured to regulate the delivery of a specific oxygen flow rate which is dependent upon the specific altitude. For example, if the altitude is determined by altimeter 10 to be at or below a certain predetermined threshold, then controller 20 can send a wired or wireless signal to the oxygen delivery components to deliver supplemental oxygen at or above a specific flow rate. Alternatively, controller 20 can send a wired or wireless signal to the oxygen delivery components to deliver air containing a certain percentage of oxygen.

As shown in FIG. 2, the device can also comprise a wearable helmet device 40. The wearable helmet device 40 includes, for example, a controller 20 operably connected between the SpO₂ sensor 60 and the oxygen delivery components. The controller 20 is programmed and/or configured to receive or request SpO₂ sensor data from SpO₂ sensor 60, modify or interpret that data, and either maintain the status quo or regulate oxygen delivery. The wearable helmet device 40 also includes, for example, oxygen delivery components configured and/or adapted to deliver supplemental oxygen to the wearer, including an oxygen storage component 12 to store the supplemental oxygen, a mask 80 with an oxygen delivery tube running from the oxygen storage component 12 to the user's mouth.

According to an embodiment, the wearable helmet device 40 also includes an altimeter 10 configured and/or adapted to monitor altitude of the device in aviation applications. For example, altimeter 10 is operably connected to the controller 20, which is programmed and/or configured to receive or request sensor data from altimeter 10, modify or interpret that data, and either maintain the status quo or regulate oxygen delivery.

According to an embodiment, a wearable device 40 also includes one or more speakers for communication, and/or to provide an audible hypoxia warning to the user. For example, upon detection of a hypoxic state by SpO₂ sensor 60, or upon detection of a certain altitude which could lead to a hypoxic state, the controller 20 can be programmed and/or configured to activate a warning signal to the user that supplemental oxygen is necessary. The controller 20 can also be programmed and/or configured to activate a warning signal to the user that supplemental oxygen is being delivered, or that delivery is being ceased. The wearable device 40 may also include manual controls and a user interface such that the user can manually override the actions of controller 20 and wearable device 40.

Therefore, according to an embodiment of wearable device 40, oxygen is automatically directed at the face when hypoxia is detected to return the user from a state of incapacitation, thereby providing the user with the cognitive ability to take follow-on corrective actions. According to an embodiment, the implementation of the emergency oxygen system would involve an alarm to allow the user to override the system.

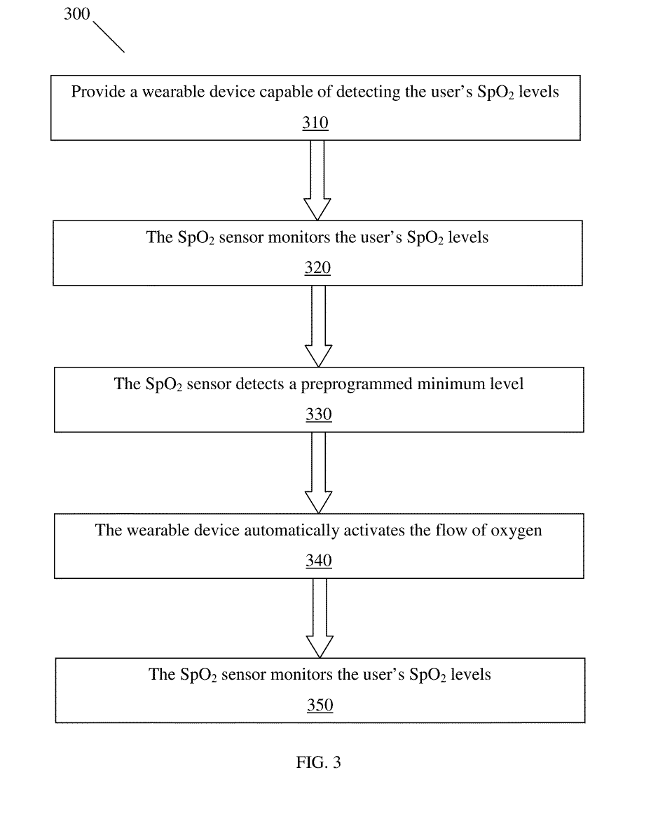

Depicted in FIG. 3 is a method 300 for providing supplemental oxygen upon detection of a hypoxic and/or potentially hypoxic state of a user. In step 310, the user puts on or activates a wearable device 40, which is configured according to any of the embodiments described herein, or as otherwise envisioned herein. For example, the user's wearable device 40 can include, among other things, a controller 20 operably connected between the SpO₂ sensor 60 and an oxygen storage component 12 to store the supplemental oxygen, and a boom 50 or mask 80 with an oxygen delivery tube running from the oxygen storage component 12 to the user's mouth. The wearable device may also include an altimeter 10 operably connected to controller 20 and configured to monitor altitude of the device in aviation applications.

In step 320, the wearable device 40 monitors the user's SpO₂ levels utilizing the SpO₂ sensor. The SpO₂ sensor can monitor the user's SpO₂ levels continuously or periodically. According to one embodiment, the SpO₂ sensor can monitor the user's SpO₂ levels continuously but only send a signal to the controller 20 when a certain threshold has been reached. Alternatively, the SpO₂ sensor can monitor the user's SpO₂ levels and continuously send that information to controller 20.

In step 330, the wearable device 40 detects that the user's SpO₂ levels have reached a predetermined minimum. For example, the SpO₂ sensor can monitor the user's SpO₂ levels and send a wired or wireless signal to controller 20 that a threshold has been reached. In another embodiment, the SpO₂ sensor monitors the user's SpO₂ levels and continuously or periodically sends that information to controller 20, which compares the data to a predetermined minimum or range to determine if the received data matches or varies from that predetermined minimum or range.

In step 340, the controller 20 sends a wired or wireless signal that causes the flow of supplemental oxygen to begin. For example, controller 20 can send a signal to oxygen storage component 12 to start the flow of oxygen through the oxygen delivery components to the user's nose and/or mouth. In one embodiment, the controller 20 sends a signal to open a valve if oxygen is needed, or a signal to close the valve if oxygen is no longer needed. The controller 20 can also control the flow rate of the oxygen once it is activated. This automated backup oxygen system therefore provides emergency oxygen when the system detects a hypoxic state.

Alternatively, the controller 20 can send a wired or wireless signal that causes the flow of an existing oxygen supply to increase or decrease, or adjusts the mixture ratio of delivered gas, or adjusts the oxygen pressure, or makes one or more of a number of other changes in order to remedy the hypoxic state. For example, especially in a hospital, ambulance, or home setting, it may be necessary to adjust the flow, ratio, and/or pressure of oxygen delivered to an individual rather than simply turn the oxygen on or off.

According to another embodiment, the wearable device 40 can also be programmed and/or configured to provide the user with an audio warning that onset of hypoxia has been detected and that emergency oxygen is about to be delivered unless another corrective action is taken.

In step 350, the SpO₂ sensor continues to monitor the user's SpO₂ levels in order to determine whether the levels return to or exceed the predetermined minimum. If they do, the controller 20 can diminish or stop the flow of supplemental oxygen to the user. Similarly, if the wearable device 40 is monitoring altitude instead of or in addition to SpO₂ levels, the controller 20 can diminish or stop the flow of supplemental oxygen to the user if a certain altitude is reached. Therefore, according to an embodiment, a barometric pressure sensor allows the system to detect changes in altitude as an additional feedback tool for the system to apply the hypoxia detection algorithms. This could be used, for example, in aviation to allow the system to automatically obtain a baseline SpO₂ level for the individual before takeoff thus improving the system's ability to determine the SpO₂ level that corresponds with onset of hypoxia.

Although the present invention has been described in connection with a preferred embodiment, it should be understood that modifications, alterations, and additions can be made to the invention without departing from the scope of the invention as defined by the claims.

What is claimed is:

1. A wearable hypoxic state detection device, the device comprising:
an SpO₂ sensor configured to measure a user's oxygen saturation;
an oxygen reservoir;
an oxygen conduit positioned to deliver oxygen from said oxygen storage reservoir to the user's inhalation flow path; and
a controller operably connected between said SpO₂ sensor and said oxygen delivery component, wherein said controller is configured to automatically induce or modify a flow of oxygen from said oxygen reservoir through said oxygen conduit when a predetermined oxygen saturation level is detected by said SpO₂ sensor.

2. The wearable hypoxic state detection device of claim 1, wherein the modification is an adjustment of the flow rate of the oxygen, an adjustment of a ratio of oxygen to total gas delivered to the user, or an adjustment of the pressure of the oxygen.

3. The wearable hypoxic state detection device of claim 1, wherein said SpO₂ sensor is a reflectance sensor.

4. The wearable hypoxic state detection device of claim 1, wherein said SpO₂ sensor is a transmittance sensor.

5. The wearable hypoxic state detection device of claim 1, wherein said controller is configured to automatically stop the flow of oxygen from said oxygen reservoir through said oxygen conduit when a predetermined oxygen saturation level is detected by said SpO₂ sensor.

6. The wearable hypoxic state detection device of claim 1, further comprising a microphone.

7. The wearable hypoxic state detection device of claim 1, further comprising a speaker.

8. The wearable hypoxic state detection device of claim 1, wherein the device is at least substantially worn on the user's head.

9. The wearable hypoxic state detection device of claim 1, wherein said oxygen conduit is situated within a mask.

10. The wearable hypoxic state detection device of claim 1, wherein said controller is operably connected to an oxygen flow valve.

11. The wearable hypoxic state detection device of claim 1, further comprising a communications module.

12. The wearable hypoxic state detection device of claim 1, further comprising an altimeter operably connected to said controller.

13. A hypoxic state detection system, the system comprising:
an oxygen reservoir; and
a wearable hypoxic state detection device comprising: (i) an SpO₂ sensor configured to measure a user's oxygen saturation; (ii) an oxygen conduit positioned to deliver oxygen from said oxygen storage reservoir to the user's inhalation flow path; and (iii) a controller operably connected between said SpO₂ sensor and said oxygen delivery component, wherein said controller is configured to automatically induce or modify a flow of oxygen from said oxygen reservoir through said oxygen conduit when a predetermined oxygen saturation level is detected by said SpO₂ sensor.

14. The system of claim 13, wherein said SpO₂ sensor is a reflectance sensor.

15. The system of claim 13, wherein said SpO₂ sensor is a transmittance sensor.

16. The system of claim 13, wherein said controller is configured to automatically stop the flow of oxygen from said oxygen reservoir through said oxygen conduit when a predetermined oxygen saturation level is detected by said SpO₂ sensor.

17. The system of claim 13, further comprising a microphone.

18. The system of claim 13, wherein the wearable hypoxic state detection device is at least substantially worn on the user's head.

19. The system of claim 13, further comprising an altimeter operably connected to said controller.

20. A wearable hypoxic state detection device configured to be worn at least substantially on a user's head, the device comprising:
an altimeter;
an SpO₂ sensor configured to measure a user's oxygen saturation;
an oxygen reservoir;
an oxygen conduit positioned to deliver oxygen from said oxygen storage reservoir to the user's inhalation flow path; and
a controller in communication with said altimeter, and operably connected between said SpO₂ sensor and said oxygen delivery component, wherein said controller is configured to automatically induce or modify a flow of oxygen from said oxygen reservoir through said oxygen conduit when a predetermined oxygen saturation level or altitude is detected by said SpO₂ sensor, and wherein said controller is further configured to automatically stop the flow of oxygen from said oxygen reservoir through said oxygen conduit when a predetermined oxygen saturation level or altitude is detected by said SpO₂ sensor.
